(19) United States
(12) Patent Application Publication
(10) Pub. No.: US 2025/0134382 A1
(43) Pub. Date: May 1, 2025

(54) EXAMINATION DEVICE FOR OPTICAL MEDICAL EXAMINATIONS

(71) Applicant: HEINE Optotechnik GmbH & Co. KG, Gilching (DE)

(72) Inventors: Felix MICHEL, Gilching (DE); Holger HEIN, Gilching (DE)

(21) Appl. No.: 18/931,641

(22) Filed: Oct. 30, 2024

(30) Foreign Application Priority Data
Oct. 31, 2023 (DE) 10 2023 130 164.0

(51) Int. Cl. A61B 5/00

(52) U.S. Cl. CPC A61B 5/0077; A61B 5/004

(57) ABSTRACT

An examination device for optical medical examinations, in particular a dermatoscope, including a camera unit for taking at least one image of an examination region of a patient, a display device and a control unit. The camera unit includes a sensor element, a lens and an adjustment device for the lens. The lens is located in an observation light path of the camera unit in front of the sensor element. The adjustment device moves the lens along the optical axis of the lens into different positions to bring into focus different examination planes in the examination region. The control unit controls the adjustment device and is connected to this. Information on two or more predefined examination planes in the examination region is stored in the control unit with the corresponding positions of the lens. The display device shows a representation of at least one image taken by the camera unit.

### FIELD OF THE DISCLOSURE

The disclosure relates to an examination device for optical medical examinations, in particular a dermatoscope.

### BACKGROUND

Optical examination devices, in particular dermatoscopes, are known and usually comprise an optics system as well as an illumination device, with which an examination region can be illuminated. In addition, optical examination devices are available that have an integrated camera which is configured to take images of the examination region and to display these on a display of the optical examination device.

Different sites of the patient's body that are to be examined differ with regard to the type and extent of the desired examination region. For example, sublayers of the human skin, such as the epidermis and the dermis, have different thicknesses depending on whether the section of skin viewed is situated on the breast, head or back of the hand of the patient in question. If a certain sublayer of the skin is to be viewed by means of the optical examination device, it must therefore be capable of adapting to which sublayer of the skin is brought into focus by means of the optical system and is thus capable of being viewed in a display as in-focus as possible.

For this purpose, it is known that an operator of the examination device, in particular a doctor, can manually adjust the focus of the camera to bring into focus the desired layer of the skin in the examination region to be viewed.

This process is however linked to considerable effort for the operator of the examination device and extends the examination times, thereby allowing less time for patient consultations.

### SUMMARY

There is a need to provide an examination device for optical medical examinations that is simple to operate and enables, in particular, images of an examination region to be obtained in high quality in a simple and reproducible way.

For this purpose, an examination device for optical medical examinations, in particular a dermatoscope, is provided comprising a camera unit for taking at least one image of an examination region of a patient, a display device and a control unit, wherein the camera unit comprises a sensor element, a lens and an adjustment device for the lens, and wherein the lens is located in an observation light path of the camera unit in front of the sensor element. The adjustment device is configured to move the lens along the optical axis of the lens into different positions to bring into focus different examination planes in the examination region. The control unit is configured to control the adjustment device and is connected to this, and information on two or more predefined examination planes in the examination region is stored in the control unit with the corresponding positions of the lens. The display device is configured to show a representation of at least one image taken by the camera unit.

The disclosure is based on the notion of providing an examination device that enables at least partially automated, preferably fully automated adjustment of the lens in the observation light path through specified or predefined positions so that different examination planes can also be examined without manually adjusting the examination device. In addition, it ensures that a desired examination plane in the examination region is displayed in focus.

As the predefined examination planes are linked to the corresponding positions in the control unit and simultaneously the control unit is configured to control the adjustment device, it is ensured that the lens is moved into the correct position through the selection of an examination plane by the operator of the examination device, in said position the desired examination plane is in focus.

The term “examination region” refers to here and in the following, for example, a three-dimensional section of the patient to be examined which can be understood as a sequence of superimposed examination planes or sections.

The position of the lens related to the corresponding examination plane is determined particularly in such a way that the lens images the examination plane in focus on the sensor element when the lens is in the position related to the examination plane.

The information on the examination plane that is stored in the control unit is, in particular, the depth of the corresponding examination plane in the patient's skin, for example defined as the distance of the corresponding examination plane from the skin surface of the patient, wherein the skin surface is defined above the stratum disjunctum, thus above the outermost part of the horny layer of the epidermis.

The examination device is, for example, a handheld device.

For example, the examination region is a region of the patient's skin. Accordingly, the examination device can be a dermatoscope for examining the region of the patient's skin. In applications such as this, it is particularly advantageous that the examination device can be brought in focus at least in a partially automated manner to simply ensure depending on the region of the patient's skin to be examined that the operator of the examination device obtains an in-focus image of the corresponding examination plane.

Accordingly, the information on the examination planes can contain information on the skin type, the body part, the type of skin region, the type of suspected lesion and/or the position of a skin region to be examined.

The type of the skin region includes, for example, the ethnicity, whether the skin is male or female or is diseased or healthy and/or whether there is mucous membrane or not.

The body part can be, for example, the patient's face, upper body, abdomen, back, hand or foot.

The position of the skin region to be examined is defined, in particular, through the site on the corresponding body part. For example, the position indicates that the skin region is part of the dorsum of the hand or foot.

In a variant, the examination device comprises a user interface with which the skin type, the body part to be examined, the type of skin region, the type of suspected lesion and/or the position of the skin region to be examined is selectable, and wherein the predefined examination planes are selected depending on said selected at least one skin type, the body part to be examined, the type of skin region, the type of suspected lesion and/or the position of the skin region to be examined. In this way, the user of the examination device must only specify the planned application site via at least one of the previously defined parameters via the user interface to put the corresponding examination plane into focus and does not have to undertake any manual adjustments.

In particular, in this way, intuitive use of the examination device is enabled as the user does not have to enter any information on abstract or numerical values for the position or depth of the desired examination plane in the examination region via the user interface, but can use simply understood terms for selecting the desired examination plane.

To simplify the operation of the examination device still further, the number of available options via the user interface can be limited. For example, only a limited number of standard settings are offered via the user interface that correspond to preset measurement scenarios.

For example, the standard settings can specify the measurement scenarios “face”, “upper body”, “dorsum of the hand” and “muscosa”, each in a variant for male and female patients.

Preferably, the examination device is configured to recognise and/or select the skin type, the body part to be examined, the type of skin region, the type of suspected lesion and/or the position of the skin region to be examined. In this way, the operation of the examination device is simplified even further as the operator of the examination device no longer has to configure the examination region personally via the user interface. In other words, the examination device brings an examination plane to be examined into focus preferably in an at least partially automated manner, in particular fully automated manner.

Preferably, the examination device is configured to recognise and/or select the skin type, the body part to be examined, the type of skin region, the type of suspected lesion and/or the position of the skin region to be examined by means of an image taken by the camera unit or an image taken by a second camera unit of the examination device.

For example, the examination device, in particular the control unit, preferably recognises the skin type, the body part to be examined, the type of skin region, the type of suspected lesion and/or the position of the skin region to be examined by means of an image taken, which has been taken by means of the camera unit that was focussed on the exterior surface of the contact plate or the skin surface.

The second camera unit comprises in this context in particular a viewing direction that differs from the first camera unit.

Said at least one skin type can be predetermined through the body part, the type of skin region, the type of suspected lesion and/or the position of the skin region on the patient. In other words, the skin type can be determined by at least one further information on the examination plane.

The adjustment device can be an actuator, in particular a stepper motor or a voice-coil motor. Such an adjustment device enables particularly precise control over the position of the lens along the optical axis, has small installation space requirements and is available economically.

In a further embodiment, the examination device comprises a user interface, with which one of the predefined examination planes is selectable, and wherein the control unit is configured to move the lens into the position related to the selected examination plane, in particular wherein only such predefined examination planes are offered and/or are selectable for selection via the user interface, which are allocated to the previously selected skin type, body part, type of suspected lesion, type and/or position of a skin region to be examined. In this way, particularly simple operation of the examination device is enabled in which the operator is offered to select the examination plane directly, wherein in particular merely that subselection of the examination plane is shown that corresponds to a restriction already undertaken previously by the operator or by the examination device itself.

The user interface can comprise at least one button and/or a touch-sensitive display for interaction with the operator of the examination device.

The user interface for selecting one of the predefined examination planes is, in particular, the same user interface with which the skin type, the body part to be examined, the type of skin region, the type of suspected lesion and/or the position of the skin region to be examined is selectable.

The display device is configured to show a representation of at least one image taken by the camera unit as a live representation. Accordingly, the examination device is in particular a digital dermatoscope.

If the user interface comprises a touch-sensitive display, the touch-sensitive display can be part of the display device. In this way, a particularly compact construction of the examination device is realised.

In a further variant, the examination device comprises a user interface, by means of which the position of the lens is settable in such a way that the position of the lens is changed from the current position by a predefined value or a multiple of the predefined value. In this way, additional examination planes are focusable about the predefined examination plane, wherein the corresponding additional examination planes are arranged at fixed distances about the predefined examination plane. This enables the operator of the examination device to gain a three-dimensional insight of the area closely surrounding the examination region around the predefined examination plane.

The distance of the additional examination planes defined by the predefined value or the multiple of the predefined value is, in particular, smaller than the distance between the examination planes predefined by the different positions. In other words, a superordinate selection of the predefined examination planes is possible through the predefined positions, while the adjustment via the user interface enables a fine adjustment around these predefined examination planes.

In yet another variant, the position of the lens is manually continuously adjustable via the user interface. In other words, in this embodiment, the position of the lens is also adjustable without the examination planes or information on the examination plane being specified by the operator in advance. This enables the operator, in particular an operator experienced in using the examination device, to carry out a needs-based and targeted control of the examination plane in focus, without being restricted by potential information and values stored in the control unit and without impairing the particularly simple basic functionality of the examination device.

The user interface for changing the position of the lens is, in particular, the same user interface that has already been described for selecting the predefined examination plane as well as the skin type, the body part to be examined, the type of skin region, the type of suspected lesion and/or the position of the skin region to be examined.

To increase the quality of the images obtained by means of the examination device further, the control unit can be configured to determine said two or more positions using autofocusing in a calibration mode, in which the examination device is placed on a reference surface, and/or to carry out a white balance using at least one image of the reference surface taken by means of the camera unit.

In this context, the position for the calibration can deviate from the position related to the examination planes, wherein the position related to the examination planes is determined in the travel of the lens starting from the position for the calibration through a conversion of the known differential values of the examination planes to a calibration plane, in which the reference surface is located.

The reference surface contains in particular a target that is used for the autofocusing and/or the white balance. Through the autofocusing, the position of the reference surface or calibration plane is determined which is regarded as skin surface in this context. The autofocusing is determined, in particular, through a determination of the maximum contrast in the image taken by the camera unit depending on the position of the lens.

It is also conceivable that autofocusing occurs in the known way using the phase shift on the cells of the sensor element (what is termed PDAF—phase detection autofocus).

By executing a white balance in the calibration mode, a particularly reliable colouring of the images obtained while using the examination device is enabled. For example, the ageing of the device is balanced by the white balance. This is particularly significant when examining regions of a patient's skin.

The reference surface is, for example, the surface of a calibration card.

For this purpose, also a method for capturing an image of an examination region by means of an examination device, in particular an examination device as described previously, is provided comprising: an examination plane is selected and a lens of a camera unit of the examination device is adjusted to one of the positions allocated to the selected examination plane. Subsequently, an image is taken by means of a sensor element of the camera unit.

The image taken may than be stored and/or displayed as a live representation. In particular, in the case of a live representation, permanent storage is not necessary.

The selection of the examination plane takes place by means of the operator or the examination device. In other words, the examination plane can be selected either based on a specification or input of the operator or at least in part in an automated manner by the examination device.

In this context, reference is made to the above embodiments for the examination device, whose features and characteristics also apply to the method according to the disclosure and vice versa.

Additional features and advantages of the disclosure are found in the following description as well as the attached drawings to which reference is made. In the drawings:

### BRIEF DESCRIPTION OF THE DRAWINGS

FIG. 1 shows an examination device according to an embodiment of the disclosure in a perspective view,

FIG. 2 shows a further perspective view of the examination device according to FIG. 1,

FIG. 3 shows a schematic section view of selected parts of the examination device according to FIG. 1,

FIG. 4 shows a first representation of the examination device in FIG. 1 in a method according to the disclosure for capturing an image of the examination region,

FIG. 5 shows a representation analogous to FIG. 4, wherein another examination region is viewed,

FIG. 6 shows a second representation of the examination device analogous to FIG. 4,

FIG. 7 shows a schematic representation of a reference surface for calibrating the examination device from FIG. 1, and

FIG. 8 shows a block diagram of the method according to the disclosure.

### DETAILED DESCRIPTION

Lists having a plurality of alternatives connected by “and/or”, for example “A, B and/or C” are to be understood to disclose an arbitrary combination of the alternatives, i.e. the lists are to be read as “A and/or B and/or C” or as “at least one of A, B or C”. The same holds true for listings with more than three items.

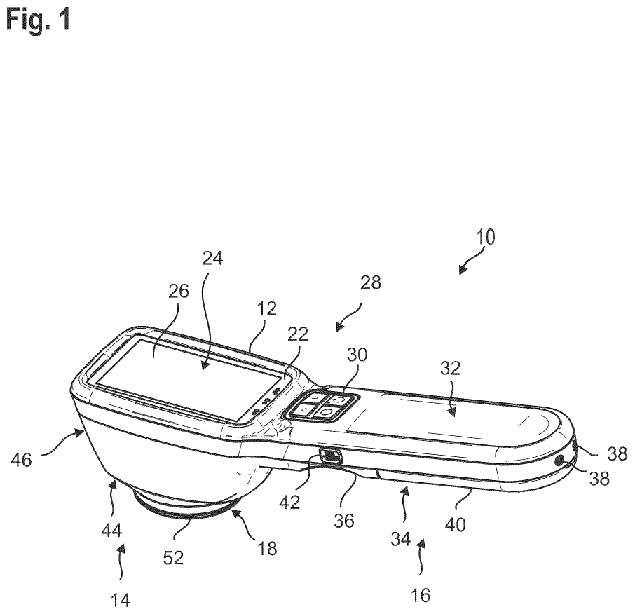

In FIG. 1, an examination device 10 for optical medical examinations is shown.

The examination device 10 is a dermatoscope, for example, and is designed as a handheld device in the shown embodiment.

The examination device 10 comprises a housing 12 and has a head region 14 and a grip region 16 which adjoins the head region 14.

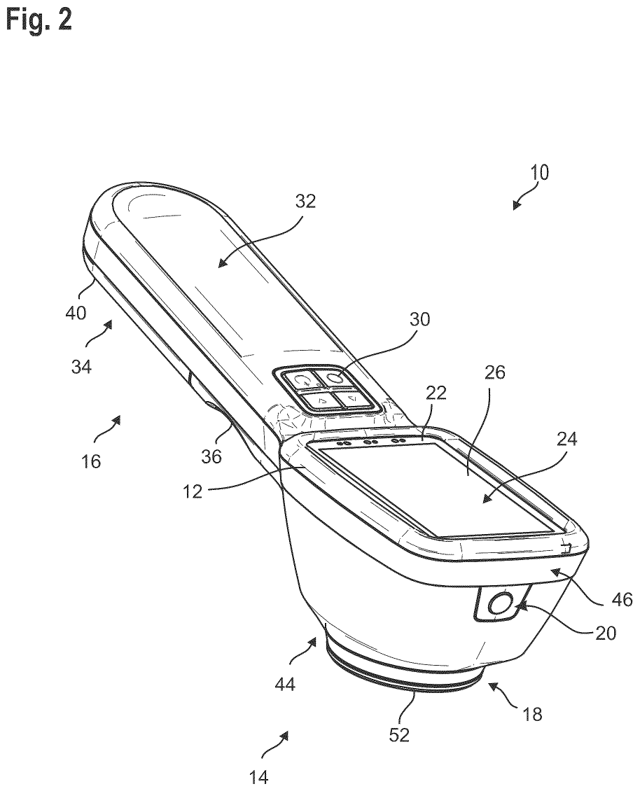

A camera unit 18 as well as a second camera unit 20 (cf. FIG. 2), which will be discussed in more detail later, are located in the head region 14. The second camera unit 20 is however optional.

Furthermore, the examination device 10 has a display device 22 comprising a display 26 located on the upper side 24 of the head region 14. The display 26 is configured to display images taken by the camera unit 18, in particular in the form of a live display.

The grip region 16 comprises a user interface 28 with control elements 30 that are located in the form of buttons on the upper side 32 of the grip region 16.

It is conceivable that the control elements 30 are also designed differently, for example, as a touch-sensitive display. The display device 22, namely the display 26, can also be designed as a touch-sensitive display that is simultaneously part of the user interface 28. In this case, it is also conceivable that control elements 30 in the form of buttons can be dispensed with.

The control elements 30 are located along the grip region 16 on the end of the grip region 16 facing towards the head region 14 so that an operator of the examination device 10 can operate these, for example, with the thumb when grasping the grip region 16 with the hand.

In addition, a tapering section 36 is provided on the underside 34 of the grip region 16, said tapering section 36 being shaped in such a way that the operator can insert, for example, the index figure in the tapering section 36 when the grip region 16 is being grasped by the hand. In this way, the hand position when using the examination device 10 is particularly ergonomic.

Moreover, a charging interface 38 is provided in FIG. 1 on the end of the grip region 16 facing away from the head region 14. The charging interface 38 is used to electrically charge an energy storage device integrated within the housing 12 (not shown).

For example, the examination device 10 is part of an examination device assembly that comprises the examination device 10 and a charging station for the examination device 10. In this case, the charging station can comprise a receiving space, into which the examination device 10 is inserted detachably in a non-destructive manner with the end of the grip region 16 having the charging interface 38 in order to execute a charging process.

Fundamentally, it is also possible that instead of a rechargeable energy storage device, thus an accumulator, that a single-use battery is provided as replaceable energy storage device.

To enable a replacement of the integrated energy storage device, the grip region has a non-destructive detachable covering element 40 that is attachable, for example, on the housing 12 via a snap-fit connection. By removing the covering element 40, the replaceable energy storage device can be removed and replaced.

In FIG. 1, it is also evident that a communication interface 42, designed as a USB C interface in the shown embodiment, is provided in the grip region 16. Via the communication interface 42, the examination device 10 can be connected to an external computer-aided data evaluation system, in particular to transmit images taken by the camera unit 18 and/or the second camera unit 20.

It is conceivable that the communication interface 42 is also designed differently, for example as an interface for wireless communication.

Moreover, the communication interface 42 can be connected to the integrated energy storage device electrically conductively so that the integrated energy storage device is also electrically chargeable via the communication interface 42.

Moreover in FIG. 1, it is evident that the grip region 16 is moulded under an angle on the head region 14. The planes that are defined by the upper side 24 of the head region 14 and the upper side 32 of the grip region 16 are at an angle to each other. In this way, the ergonomics of the examination device 10 are improved further.

FIG. 2 shows a further perspective view of the examination device 10, in which the arrangement of the camera unit 18 and the second camera unit 20 can be seen better.

The camera unit 18 is allocated to the underside 44 of the head region 14, namely integrated into the underside 44. The integration is in such a way that the viewing direction of the camera unit 18 extends downwards, in the shown embodiment thus substantially perpendicular to the upper side 24 of the head region 14.

In contrast, the second camera unit 20 is allocated to a front side 46 of the head region 14, namely integrated into the front side 46. The integration is in such a way that the viewing direction of the second camera unit 20 extends forwards, thus substantially parallel or slightly inclined to the upper side 24 of the head region 14.

Fundamentally, the examination device 10 can just comprise the camera unit 18, thus no second camera unit 20.

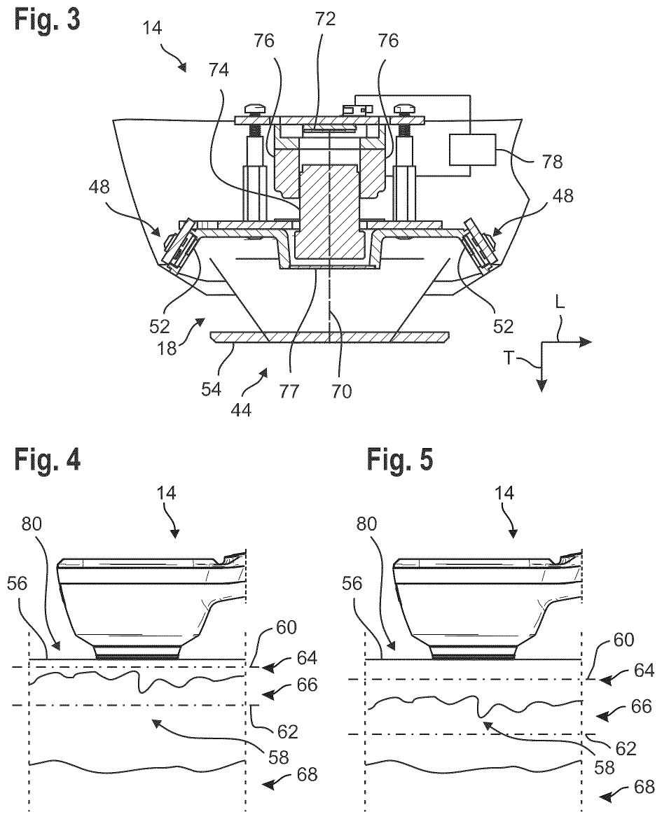

FIG. 3 shows a schematic section view of selected parts of the examination device 10, especially the head region 14, to illustrate the construction and the functioning of the camera unit 18 further. The section plane in FIG. 3 runs parallel to a plane that is defined by the longitudinal direction L of the examination device 10 and a depth direction T that is perpendicular to the longitudinal direction L.

The camera unit 18 comprises several lighting devices 48 that are designed, for example, as light-emitting diodes (LEDs) and are used to emit light in the direction of a contact plate 54.

The light emitted by the lighting devices 48 is light of a predefined wavelength or a predefined wavelength spectrum.

In addition, polarization filters 52 are provided between several of the lighting devices 48 and the contact plate 54.

The light devices 48 provided with polarization filters 52 can be connected independently of the lighting devices 48 without polarization filters 52 so that the examination region can be illuminated with polarized light and non-polarized light as desired.

At the underside 44 of the head region 14, the camera unit 18 borders with the contact plate 54 which during use of the examination device 10 is placed on the skin surface 56 of a patient to be examined (cf. FIG. 4).

Through the positioning of the contact plate 54 on the patient, a patient's examination region 58 is defined that comprises a plurality of successive examination planes along the depth direction T, starting from skin surface 56, of said examination planes only two predefined examination planes 60 and 62 are indicated with dashed lines in FIG. 4 for the sake of clarity.

The examination region 58 extends from the skin surface 56 that is defined over the stratum disjunctum, and rests on the contact plate 54, through the epidermis 64 to the dermis 66. The subcutis 68 is located below the dermis 66, said subcutis 66 does not usually have to be viewed, however can be optionally also part of the examination region 58.

The light emitted by the lighting devices 48 penetrates through the contact plate 54 into patient's skin, especially in the examination region 58, and is reflected by this at least in part in the direction of the camera unit 18 along an observation light path 70 (cf. FIG. 3).

The observation light path 70 comprises, for example, all light that reaches the sensor element 72.

To detect this reflected light, the camera unit 18 has a sensor element 72 as well as a lens 74, wherein the lens 74 is located along the observation light path 70 in front of the sensor element 72 and is used to focus incident light onto the sensor element 72.

The sensor element 72 is, for example, a CCD sensor or a CMOS sensor.

Moreover, the lens 74 is connected to an adjustment device 76 that is configured to move the lens 74 along the optical axis of the lens 74 into different positions, i.e. in the axial direction of the lens 74.

The adjustment device 76 is a voice-coil motor in the shown embodiment. Fundamentally, however, another type of actuator can also be used as an adjustment device, for example a stepper motor.

A lens polarisation filter 77 is also arranged between the skin surface 56 and the lens 74 and is used to block undesirable elements of the light reflected from the examination region 58. In particular, the polarization direction of the lens polarization filter 77 is perpendicular to the polarization direction of the polarization filters 52 of the lighting devices 48.

As the lens 74 can be moved by the adjustment device 76, the lens 74 can occupy various positions along the optical axis. Thus, in the case of the same lens 74, depending on the current position of the lens 74, another examination plane in the examination region 58 can be imaged by the lens 74 in focus onto the sensor element 72. In other words, the position defines the current examination plane of the camera unit 18.

The adjustment device 76 is connected to the control unit 78 in a signal-transmitting manner, said control unit 78 being configured to control the adjustment device 76 and thus determine the position of the lens 74.

Information on two or more predefined examination planes in the examination region is stored in the control unit 78, for example, the predefined examination planes 60 and 62 shown in FIG. 4, each with corresponding positions of the lens 74.

Moreover, the control unit 78 is connected to the user interface 28 of the examination device 10 in a signal-transmitting manner. Thus, the position of the lens 74 can be set by the operator of the examination device 10 via the user interface 28, and thus the control unit 78 and the adjustment device 76.

Furthermore, the control unit 78 is connected to the sensor element 72 in a signal-transmitting manner so that the sensor element 72 can transmit images captured by the camera unit 18 to the control unit 78. The images obtained by the camera unit 18 can then be stored in the control unit 78 and transmitted to an external device via a communication interface 42.

In the following, the functioning of the examination device 10 is described in more detail in relation to a method according to the disclosure for capturing at least one image of the examination region 58.

In FIG. 4, a first application scenario of the examination device 10 is shown schematically.

The examination device 10 is placed in a skin region 80 on the skin surface 56 of a patient to be examined, wherein the contact plate 54 touches the skin surface 56. It is also possible that a contact liquid is applied between the skin surface 56 and the contact plate 54, in particular a dermatoscopy oil, that serves to match the refractive indexes of the camera unit 18 and the skin surface 56 of the patient.

The examination region 58 defined by the selected contact position on the skin surface 56 comprises a sequence of examination planes characteristic for the corresponding site on the patient, said examination planes being arranged one above the other along the depth direction T.

Thus, information on the two predefined examination planes 60 and 62 is stored in the control unit 78, wherein the predefined examination plane 60 is allocated to the epidermis 64 and the predefined examination plane 62 to the dermis 66.

The examination planes are predefined, for example, by means of their distance from the skin surface 56 in the depth direction T. For example, the examination plane “epidermis” on the dorsum of a healthy hand of a female patient is at a distance of 195 μm to the skin surface.

The dimensions of the epidermis 64 and dermis 66 on a patient however depends on each part of the patient's body to be examined so that the position of the predefined examination planes 60 and 62 along the depth direction T is also stored in the control unit 78 depending on the body part to be examined, thus is linked to further information on the predefined examination plane 60 or 62.

The information on the examination planes 60 and 62 thus comprise in particular information on the skin type, the body part, the type of skin region 80, the type of suspected lesion and/or the position of the skin region 80 to be examined.

The corresponding information on the examination plane “epidermis” of the aforementioned example thus contains the information “female” for skin type, “hand” for body part, “healthy” for type of skin region 80 and “dorsum of the hand” for position of the skin region 80 to be examined. In addition, the distance to the skin surface of 195 μm is linked to the examination plane “epidermis”, e.g. as part of the information.

For example, the skin region 80 shown in FIG. 4 is a diseased region on the back of a male patient. Such skin regions comprise a comparatively thin epidermis 64 and a comparatively thick dermis 66.

In contrast, for example, the skin region 80 shown in FIG. 5, which corresponds to a second application scenario, is a healthy region on the upper side of a male patient's hand and thus a skin region that comprises a comparatively thick epidermis 64 and a comparatively thin dermis 66 in comparison to the situation in FIG. 4.

Accordingly, different predefined examination planes 60 and 62 are stored in the control unit 78 for these different application scenarios, which ensure in each case that the examination planes 60 and 62 are in the desired region of the examination region 58 and supply a correct image.

In addition, a position of the lens 74 is stored in the control unit 78 for each of the predefined examination planes so that when selecting one of the predefined examination planes 60 or 62, the control unit 78 can move the lens 74 along the optical axis by means of the adjustment device 76 in such a way that the lens 74 in the resulting position images the corresponding examination plane 60 or 62 in focus on the sensor element 72.

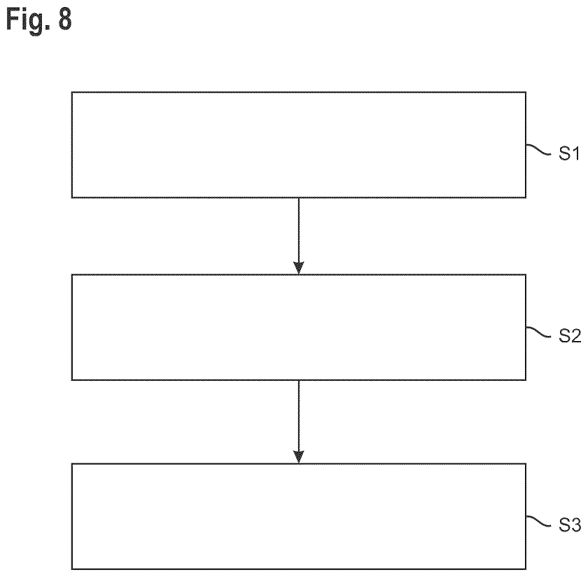

In the method according to the disclosure for capturing an image of the examination region 58, an examination plane is initially selected (step S1 in FIG. 8), wherein in particular one of the predefined examination planes 60 or 62 is selected.

This can occur by means of the operator of the examination device 10 via the user interface 28, wherein the available options are shown in particular on the display 26. For example, “dermis” and “epidermis” are offered as examination planes.

The shown options can be limited to specific application scenarios in order to simplify the operation of the examination device 10.

It is also possible that the examination plane is selected in an automated manner by the control unit 78 by placing the examination device 10 with the contact plate 54 on the skin region 80 and taking at least one image by means of the camera unit 18, by means of said image the control unit 78 determines the necessary information on the examination region 58, thus for example, which skin type, which body part, which type of skin region 80 and/or which position of the skin region 80 it is. The control unit 78 can also check a suspicion of a lesion, which can show information on the examination region 58 using the image taken.

When taking this image, the camera unit 18 is focused on the external surface of the contact plate 54 or the skin surface 56, thus brought into focus.

To this end, reference images can be stored in the control unit 78 that are compared to said at least one image. As soon as the information on the examination planes has been determined in this way in an automated manner, the operator can be presented via the user interface 28 with a selection of the predefined examination planes 60 and 62, from which it selects. Alternatively, the predefined examination plane 60 or 62 can also be selected in an automated manner by the control unit 78.

In a further embodiment, an overview image of the skin region 80, in which the examination region 58 is to be formed, can be taken by means of the second camera unit 20 that is used by the control unit 78 to determine the information of the predefined examination plane.

As soon as a predefined examination plane has been selected, for example the predefined examination plane 60, the control unit 78 sends an setting signal to the adjustment device 76 that in turn moves the lens 74 of the camera unit 18 along the optical axis to a position allocated to the selected examination plane (step 2 in FIG. 8).

The allocated position is that position of the lens 74 in which the selected examination plane is imaged in focus on the sensor element 72.

Thus, at least one image of the selected examination plane, for example the predefined examination plane 60, can then be taken by means of the sensor element 72 of the camera unit 18 (step S3 in FIG. 8).

In this way, a simple and reliable option for taking patient images is possible through the allocation of the positions to the predefined examination planes and the further information linked with this, such as skin region.

The image taken can be shown to the operator on the display 26, in particular as a live display.

It is also possible that the operator sets or adjusts the position of the lens 74 and thus the examination plane 82 independently via the user interface 28, in particular one of the control elements 30.

For example, the operator can shift the examination plane by a predefined value or a multiple of a predefined value through an input on the user interface, e.g. by actuating a button. The control unit 72 then changes the position of the lens 74 by the predefined value or a multiple of the predefined value. In this way, one or more images of further examination planes 82 can be obtained that are spaced at a specified distance d₁ from a predefined examination plane. The operator is thus not limited to the predefined examination planes.

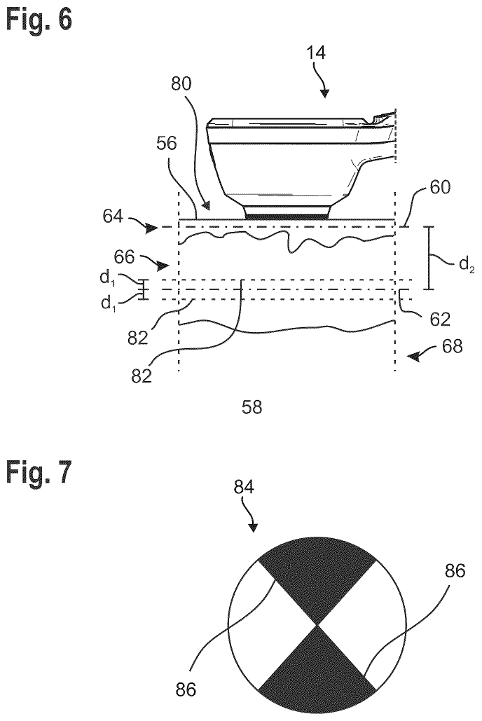

In FIG. 6, two such further examination planes 82 are indicated with dashed lines that are located about the predefined examination plane 62. In other respects, the application scenario from FIG. 6 corresponds to that from FIG. 4.

The specified distance d₁ is particularly smaller than a distance d₂ that is defined by the predefined examination planes 60 and 62.

It is also possible that the operator can execute a manual continuous adjustment of the position of the lens 74 via the user interface 28.

The examination device 10 also has a calibration mode, in which the examination device 10 is placed on a reference surface 84. In FIG. 7, an exemplary reference surface 84 is shown. The reference surface 84 is printed, for example, on a calibration card that is delivered with the examination device 10.

The reference surface 84 has at least one, preferably multiple targets 86 or contrast elements.

The examination device 10 comprising the contact plate 54 is placed on the reference surface 84 for calibration. The outer side of the contact plate corresponds to the calibration plane in this case.

The lens 74 is then moved into different positions by the control unit 78 or the adjustment device 76 until said at least one contrast element 86 is shown with a maximum contrast in the image taken of the reference surface 84.

The position for the calibration determined in this way, i.e. the position of the adjustment device 76 that is allocated to the calibration plane, is regarded as the position that is allocated to the skin surface.

Said two or more positions that are allocated to the predefined examination planes 60 and 62 can then be determined by the control unit 78.

For example, to this end, the distance to the skin surface stored for the predefined examination planes is used and the positions related to the predefined examination planes are calculated using this distance relative to the position related to the skin surface (position for the calibration).

It is also possible that position related to the skin surface determined in this way is already a position that is allocated to one of the predefined examination planes 60 and 62.

Moreover, a white balance can be undertaken using the image taken from the reference surface 84. In other words, via a comparison of the image taken from the reference surface 84 with the reference value or a reference image, a deviation of the colour representation can be determined from a desired value and stored in the control unit 78. Images obtained during use of the examination device 10 can then be corrected by means of the specific deviations in order to obtain displays of the examination region 58 that are as true in colour as possible.

Overall, the examination device 10 according to the disclosure is characterized by its simple operation and enables simultaneously images of predefined examination planes of a patient to be taken reliably.

1. An examination device for optical medical examinations comprising a camera unit for taking at least one image of an examination region of a patient, a display device and a control unit,
wherein the camera unit comprises a sensor element, a lens and an adjustment device for the lens, wherein the lens is located in an observation light path of the camera unit in front of the sensor element,
wherein the adjustment device is configured to move the lens along an optical axis of the lens into different positions in order to bring into focus different examination planes in the examination region,
wherein the control unit is configured to control the adjustment device and is connected to this, and information on two or more predefined examination planes in the examination region is stored in the control unit with related positions of the lens, and
wherein the display device is configured to show a representation of at least one image taken by the camera unit.

2. The examination device according to claim 1, wherein the examination device is a dermatoscope.

3. The examination device according to claim 1, wherein the examination region is a skin region of the patient.

4. The examination device according to claim 3, wherein the information on the examination planes contains information on at least one of a skin type, a body part, a type of the skin region, a type of suspected lesion, and a position of the skin region to be examined.

5. The examination device according to claim 4, wherein the examination device comprises a user interface with which at least one of the skin type, the body part to be examined, the type of the skin region, the type of the suspected lesion and the position of the skin region to be examined is selectable, and the predefined examination planes are selected depending on at least one of said selected at least one skin type, the body part to be examined, the type of the skin region, the type of the suspected lesion and the position of the skin region to be examined.

6. The examination device according to claim 4, wherein the examination device is configured to at least one of recognise and select at least one of the skin type, the body part to be examined, the type of the skin region, the type of the suspected lesion and the position of the skin region to be examined.

7. The examination device according to claim 6, wherein the examination device is configured to at least one of recognise and select the at least one of the skin type, the body part to be examined, the type of skin region, the type of suspected lesion and the position of the skin region to be examined by means of an image taken by the camera unit or an image taken by a second camera unit of the examination device.

8. The examination device according to claim 4, wherein at least one of said at least one skin type is given by the body part, the type of the skin region and the position of the skin region on the patient.

9. The examination device according to claim 1, wherein the adjustment device is an actuator.

10. The examination device according to claim 9, wherein the actuator is a stepper motor or a voice-coil motor.

11. The examination device according to claim 1, wherein the examination device comprises a user interface, with which one of the predefined examination planes is selectable, and the control unit is configured to move the lens into the position associated with the selected examination plane,

12. The examination device according to claim 11, wherein only such predefined examination planes are at least one of offered and selectable for selection via the user interface, which are allocated to at least one of skin type, body part, type of suspected lesion, type of a skin region and position of a skin region previously selected to be examined.

13. The examination device according to claim 11, wherein the user interface comprises at least one of at least one button and a touch-sensitive display.

14. The examination device according to claim 1, wherein the display device is configured to show a representation of the at least one image taken by the camera unit as a live representation.

15. The examination device according to claim 1, wherein the examination device comprises a user interface, by means of which the position of the adjustment device is settable in such a way that the position of the adjustment device is changed from a current position by a predefined value or a multiple of the predefined value.

16. The examination device according to claim 1, wherein the position of the adjustment device is manually continuously adjustable via a user interface.

17. The examination device according to claim 1, wherein the control unit is configured to at least one of determine said two or more positions using autofocusing in a calibration mode, in which the examination device is placed on a reference surface, and execute a white balance using at least one image of the reference surface taken by means of the camera unit.

18. A method for capturing an image of an examination region by means of an examination device comprising:
selecting an examination plane,
adjusting a lens of a camera unit of the examination device to a position allocated to the selected examination plane,
taking at least one image by means of a sensor element of the camera unit.

19. The method according to claim 18, wherein the examination plane is selected by a user or by the examination device.

20. The method according to claim 18, wherein the examination device comprises the camera unit for taking the at least one image of the examination region of a patient, a display device and a control unit,
wherein the camera unit comprises the sensor element, the lens and an adjustment device for the lens, wherein the lens is located in an observation light path of the camera unit in front of the sensor element,
wherein the adjustment device is configured to move the lens along an optical axis of the lens into different positions in order to bring into focus different examination planes in the examination region,
wherein the control unit is configured to control the adjustment device and is connected to this, and information on two or more predefined examination planes in the examination region is stored in the control unit with related positions of the lens, and
wherein the display device is configured to show a representation of at least one image taken by the camera unit.
